(19) United States
(12) Patent Application Publication
(10) Pub. No.: US 2023/0061239 A1
(43) Pub. Date: Mar. 2, 2023

(54) CURATING AN INTERFACE TERMINOLOGY FOR EFFECTIVE ANNOTATION OF ELECTRONIC HEALTH RECORDS (EHRS) OF A MEDICAL DISCIPLINE

(71) Applicant: Yehoshua Perl, Forest Hills, NY (US)

(72) Inventors: Yehoshua Perl, Forest Hills, NY (US); James Geller, West Orange, NY (US); Vipina Kuttichi Keloth, Houston, TX (US); Shuxin Zhou, Newark, NJ (US)

(73) Assignee: Yehoshua Perl, Forest Hills, NY (US)

(21) Appl. No.: 17/888,070

(22) Filed: Aug. 15, 2022

Related U.S. Application Data
(60) Provisional application No. 63/232,872, filed on Aug. 13, 2021.

(51) Int. Cl. G16H 50/70; G16H 10/60

(52) U.S. Cl. CPC G16H 50/70; G16H 10/60

(57) ABSTRACT

The technology described herein is applicable to an artifact that is a specialized interface terminology for a given medical discipline, e.g., cardiology. This interface terminology is configured to provide effective automatic annotation of EHR notes of, e.g., cardiology patients. A similar process can be applied to all types of medical specialties. Effective annotation of EHR notes will support interoperability and automatic access to the knowledge hidden in the free text in the EHR notes enabling ease of access and the ability to quickly research the same.

### CROSS-REFERENCE TO RELATED APPLICATIONS

This application claims priority to U.S. Application Ser. No. 63/232,872 filed on Aug. 13, 2021, the contents of which are herein fully incorporated by reference in its entirety.

### FIELD OF THE EMBODIMENTS

The field of the embodiments of the present application relates to annotating electronic health records (EHRs). In particular, the present application relates to curating an interface terminology for annotation of EHRs of a medical discipline.

### BACKGROUND OF THE EMBODIMENTS

Recent years have witnessed a major transformation in the field of health care, with the federal government taking the lead to encourage the use of electronic health records (EHRs) [1]. With the wide use of EHRs, large volumes of discharge summaries, lab reports, progress notes, etc. have become available to the healthcare community. Such clinical notes, specifically progress notes, contain the most up-to-date relevant information per patient.

While extremely valuable in describing the clinical conditions of a specific patient, the information is mostly recorded in unstructured text with highly specialized clinical phrases. To enable and augment interoperability and enhance healthcare quality by facilitating post hoc research studies, it would be desirable to annotate these records with concepts from a standard terminology. Without annotations, the text is often vague, ambiguous, and inadequate for automated processing. One of the important goals for converting paper records to EHRs was to facilitate annotation of EHR notes with concepts from reference terminologies to support interoperability and research. In today's EHRs, coded data entry is limited to specific segments such as problem lists and quality measures. The vision of “Meaningful Use” (MU) [2] required the use of standardized ontologies. SNOMED CT (SNOMED in short) [3], which was selected for clinical recording, is rarely utilized due to two major reasons. First, is that currently there are no satisfactory off-the-shelf tools to enable effective clinical notes annotation (covering 75-80% of the words of the text). Second, physicians record clinical notes with refined medical phrases, many of which are not contained in standard medical reference terminologies used for annotation.

Longer, refined descriptive concept phrases such as burst of atrial fibrillation and left arm phlebitis are frequently used by medical professionals in EHRs and these phrases correspond to cognitive chunks [5], which are often of higher granularity than related concepts in reference terminologies. While these chunks are constructed from simpler concepts, the emphasis is on the meaning of the entire chunk rather than the constituent concepts. Similarly, Uzuner et al. [4] report that the most challenging examples for concept extraction systems were abbreviations and descriptive concept phrases, giving the example subtle decreased flow signal within the sylvian branches for a descriptive concept phrase.

SNOMED is not sufficient to annotate cardiology clinical notes. Consider the cardiology concept supraventricular tachycardia (SVT), which is a potentially dangerous fast heart rhythm arising from the upper part of the heart. Two important types of SVT are atrioventricular nodal reentrant tachycardia (AVNRT) and atrioventricular reentrant tachycardia (AVRT). SNOMED is the most comprehensive clinical reference terminology; nevertheless, it cannot deal with this example. While SNOMED provides a code for SVT and AVRT, the latter is not classified as a child of SVT. SNOMED does not provide a code for AVNRT at all. This example illustrates that SNOMED lacks many high granularity concepts. Note that missing content in SNOMED has also led to a decrease in the inter-annotator agreement for manually annotated datasets. For example, Miñarro-Giménez et al. [6] in a study on manual annotation of clinical text with SNOMED observed that when a high granularity concept is missing, the annotators faced a dilemma of choosing the parent concept or guessing the most likely one of the many child concepts. This often led to disagreements between annotators.

Rosenbloom et al. [11] defined a clinical interface terminology as “a systematic collection of healthcare-related phrases (terms) that supports clinicians' entry of patient-related information into computer programs, such as clinical ‘note capture’ and decision support tools.” It is an interface between the users and the standard reference terminologies, required by clinical information systems [12, 13]. Interface terminologies are designed with the end-users in mind and hence consist of relatively common clinical phrases and colloquial usages as opposed to a standard concept-based aggregation of clinical information in a reference terminology.

MIMIC-III (Medical Information Mart for Intensive Care, version 3) is a freely accessible, de-identified critical care database comprising information relating to patients admitted to critical care units at the Beth Israel Deaconess Medical Center in Boston, Mass. [14]. The data is very diverse ranging from vital signs, medications, and laboratory measurements to procedure codes, diagnostic codes, billing information, and survival data. The database contains data from patients admitted to the hospital from 2001 to 2012 with a total of 61,532 intensive care unit (ICU) stays and 46,476 unique patients.

The automatic systems which can commonly be used for annotation of clinical text include the NCBO Annotator [7, 15], and the Named Entity Recognition (NER) systems MetaMap [8,16] and cTAKES [9]. The NCBO Annotator (NCBOA) (previously known as the Open Biomedical Annotator) is an ontology-based web service, available on the BioPortal platform [17], which tags biomedical text automatically with ontology terms. With NCBOA it is possible to annotate text with concepts from an ontology of the user's choice (from 830+ BioPortal ontologies). The users can also upload their own ontology to BioPortal, using BioPortal services, and utilize this ontology to annotate their text, thereby customizing the annotations based on the requirements of different studies.

MetaMap was developed by the National Library of Medicine (NLM) and can annotate clinical text with appropriate concepts from the Unified Medical Language System (UMLS) [10]. A number of options are available (e.g., source vocabulary, semantic type) that can be configured to suit various applications. cTAKES is yet another system available for the extraction of information from clinical text with the UMLS. It is available as open source software from Apache.

A literature review on clinical information extraction (IE) applications by Wang et al. [18] provides information about the frameworks, tools, and toolkits used for IE in the clinical domain. According to their review, cTAKES and MetaMap are the most frequently used tools for information extraction in the clinical domain. Both these tools use the UMLS to recognize and normalize the identified concepts [19]. The techniques used for clinical IE are mainly rule-based or machine-learning based. Another clinical NLP software system, CLAMP [20], incorporates several machine learning components and the latest release, 1.6.0, contains multiple deep learning modules.

The purpose of this invention is to describe a system and process for curating an interface terminology for providing annotation of EHRs of patients of a given medical discipline. This interface terminology is designed to support effective annotation of such EHRs covering about 75-80% of the words in the EHR text. Such performance is beyond the capability of current annotation systems and available reference terminologies.

The techniques described in this invention were described in several publications. A study of cardiology EHR notes [21] used a dataset from MIMIC-III [14]. Another study [22] of EHR notes of COVID-19 patients used the SIRM open source database from Italy [23]. Finally, a recent comprehensive study [24] of EHR notes of COVID-19 patients used the Radiopaedia opensource international database [25], from which some tables and results in this application are taken.

Review of related technology:

U.S. Pat. 11,152,084 pertains to techniques for coding a medical report that includes identifying an acronym or abbreviation in the medical report, and a plurality of phrases not explicitly included in the medical report that are possible expanded forms of the acronym or abbreviation in the medical report. From the plurality of phrases, a most likely expanded form of the acronym or abbreviation may be selected by applying to the medical report a statistical acronym/abbreviation expansion model trained on a corpus of medical reports. By applying to the medical report with the expanded acronym or abbreviation one or more statistical fact extraction models, a clinical fact may be extracted from the medical report based at least in part on the most likely expanded form of the acronym or abbreviation in the medical report, and a corresponding medical taxonomy code may be assigned to the extracted clinical fact from the medical report.

U.S. Pat. 10,755,038 pertains to methods, computer systems, and computer storage media for providing real-time analysis and annotation of clinical documents in a distributed system. A clinical transformation session is opened at a clinical transformation server maintaining sessions for one or more editors and agents operating on a clinical document. Sequences of operations on the clinical document are stored at a memory accessible by the server. At least a portion of the clinical document is analyzed in real-time to provide annotations and other document modifications to each of the one or more editors having a session at the server. Parallel annotations or modifications are resolved and a synchronized view of the clinical document is maintained based on operational transformation.

U.S. Pat. 10,509,889 pertains to a system and method utilizing deep clinical knowledge represented as a knowledge-graph to complement and enhance Natural Language Processing (NLP) for efficient and high-quality computer assisted coding of medical text. One embodiment utilizes the International Classification of Diseases version-10 Procedural Coding System (ICD-10-PCS). The system uses multiple knowledge bases combined with direct mapping provided by the ICD-10-PCS standard to enhance the coverage of assigned code. The system identifies ICD-10-PCS code considering hierarchical mapping and identifies the code by individual ICD-10-PCS character.

U.S. Pat. 9,971,848 pertains to systems and methods for producing and presenting annotations of clinical documents in a rich format are described, for instance for use with medical billing procedures. An initial XHTML document documenting a medical patient encounter and having rich formatting is used to generate a plain text document. A clinical language understanding system generates annotations, such as medical codes, which are used to annotate the XHTML document. The annotated XHTML document is then presented to a user, thus displaying for the user the annotations while retaining the rich formatting of the initial XHTML document.

As described herein, various systems and methodologies are known in the art. However, their structure and means of operation are substantially different from that of the present disclosure. At least one embodiment of this invention is presented in the drawings below and will be described in more detail herein.

### SUMMARY OF THE EMBODIMENTS

Clinical data stored in EHRs could provide valuable knowledge for research if it were annotated properly. However, almost no EHR notes are currently annotated as the performance of off-the-shelf annotation tools is unsatisfactory. The present application and its embodiments are dedicated to the annotation of EHR notes in cardiology as well as other medical specialties utilizing an interface terminology. This interface terminology is developed by the addition of high granularity concepts mined from EHR notes, to an initial version reusing SNOMED CT subhierarchies. Using text mining NLP tools with machine learning for extending this interface terminology requires proper training data.

The present application describes a complex process composed of two phases of several stages. Initially, the Cardiology Interface Terminology (CIT) is populated with the cardiology-related concepts of the SNOMED CT Reference terminology. Then the system can enhance the CIT with concepts mined from cardiology EHR notes. This ensures effective annotation of cardiology EHR notes with concepts from the CIT since the best source for concepts for annotation of EHR notes is the EHR notes.

The first phase of the process of curating a CIT uses a combination of automatic processing to mine phrases from EHR notes and reviews by domain experts for adding concepts to CIT. The automatic processing utilizes innovatively the iterative applications of so-called concatenation and anchoring operations. The resulting concepts serve as training data for the second phase. The second phase uses machine learning techniques for mining additional concepts from EHR notes after the machine learning model was trained with the above training data.

In one aspect of the present application, there is an electronic health record (EHR) annotation method, the method includes the steps of identifying one or more terms from a collection of terms, the one or more terms containing concepts relevant to at least one medical status of a patient, annotating, via a processor, a plurality of EHRs using at least two annotation processes, applying, via the processor, a difference operation to the two annotation processes to generate an initial annotation set, where the difference operation identifies one or more terms that typically appears in EHRs. Further applied are alternating stages of the concatenating and anchoring operations, via the processor, using one stage annotation set to generate a consecutive annotation set, applying, via the processor in a second phase, a machine learning algorithm, trained with concepts added during the first phase to generate a final annotation set.

The method may also include the step of automatically annotating, via a processor and using the final annotation set, one or more EHRs.

The method may also include where the concatenating step is performed before the anchoring step.

The method may also include where the concatenating and anchoring steps are repeated at least one time.

The method may also include where the concatenating step generates an initial derivative annotation set.

The method may also include where the anchoring step is performed on the initial derivative annotation set.

The method may also include where the concatenating and anchoring steps are repeated alternatingly on the derivative annotation ser resulting from the previous operation until new terms added to a generated annotation set fall below a threshold.

In another aspect of the present application there is a computer system that includes one or more processors, one or more memories, and one or more computer-readable hardware storage devices, the one or more computer-readable hardware storage devices containing program code executable by the one or more processors via the one or more memories to implement a method for annotating one or more electronic health records (EHRs), the method includes the steps of identifying one or more terms from a collection of terms, the one or more terms containing concepts relevant to at least one medical status of a patient, annotating, via a processor, a plurality of EHRs using at least two annotation processes, applying, via the processor, a difference operation to the two annotation processes to generate an initial annotation set, where the difference operation identifies one or more terms that typically appears in EHRs, applying alternative stages of concatenating and anchoring operations, via the processor, using the one stage initial annotation set to generate a consecutive annotation set, applying, via the processor in the second phase, a machine learning algorithm, trained with concepted added during the first phase to generate a final annotation set.

In yet another aspect of the present application there is a computer program embodied in a non-transitory computer-readable medium includes computer readable instructions, which when executed by a processor, cause the processor to execute a method to annotate one or more electronic health records (EHRs) includes the steps of identifying one or more terms from a collection of terms, the one or more terms containing concepts relevant to at least one medical status of a patient, annotating, via a processor, a plurality of EHRs using at least two annotation processes, applying, via the processor, a difference operation to the two annotation processes to generate an initial annotation set, where the difference operation identifies one or more terms that typically appears in EHRs, applying alternating stages of concatenating and anchoring operations, via the processor, using one stage set to generate a consecutive annotation set, applying, via the processor, a machine learning algorithm, trained with concepts added during the first phase to generate a final annotation set.

Other technical features may be readily apparent to one skilled in the art from the following figures, descriptions, and claims.

### BRIEF DESCRIPTION OF THE DRAWINGS

To easily identify the discussion of any particular element or act, the most significant digit or digits in a reference number refer to the figure number in which that element is first introduced.

FIG. 1 is an excerpt from an exemplary cardiology EHR, annotated by the initial CIT (ICIT) (light grey) and the concepts obtained by the DIFF operation (dark grey).

FIG. 2 is an example of concatenation and anchoring in accordance with an embodiment of the present application. Annotated concepts are highlighted in light or dark grey, overbars mark concatenation, and underlines mark anchoring. (highlights with light and dark coloring are used to distinguish between some consecutive annotated phrases).

FIG. 3 illustrates a flow chart for phase one implementation of an embodiment of the present application.

FIG. 4 illustrates a flow chart for phase two implementation of an embodiment of the present application.

FIG. 5A is an exemplary clinical note annotated by CIT. Annotated phrases are highlighted alternatingly with light and dark grey colors to distinguish between consecutive annotated phrases.

FIG. 5B is an exemplary clinical note annotated by CIDO. Annotated phrases are highlighted alternatingly with light and dark grey colors to distinguish between consecutive annotated phrases.

FIG. 5C is an exemplary clinical note annotated by SNOMED. Annotated phrases are highlighted alternatingly with light and dark grey colors to distinguish between consecutive annotated phrases.

FIG. 6 is a block diagram of a computing device used within a system, according to at least some embodiments of the present application disclosed herein.

### DETAILED DESCRIPTION OF THE EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

Described herein is a knowledge base containing concepts of a medical field with hierarchical and lateral (semantic) relationships connecting them.

As used herein, “annotation of text” means matching words or phrases in the text to Concepts from a Terminology. An annotated text enables one to perform research on the subject matter described in the text. It is also easier to achieve interoperability between different systems with annotated text.

Electronic Health Records (EHRs) contain, amongst other information, text blocks written by medical doctors (MDs), describing the symptoms, diagnoses, treatments, and course of the disease of a patient. The MDs also fill in boxes with specific data values, which constitute the structured part of the EHR (in addition to the text blocks). While researchers can conduct research using the structured part of an EHR, it is difficult to conduct research based on textual EHR notes. Since the text of the EHR contains the best clinical description of the treatment and the course of the disease, it is crucial to provide researchers with annotations of the text blocks in EHRs. Currently, almost no EHRs in hospitals or clinics are annotated. The reason is that efforts to annotate EHRs using existing software annotators or natural language processing (NLP) techniques utilizing a reference terminology such as SNOMED CT did not provide effective annotations. Alternatively, manual annotation is difficult and expensive. As a result, existing EHRs are not annotated.

The lack of EHR annotations prevents medical research and thus medical progress. This situation was recently demonstrated with the COVID-19 pandemic where MDs could not discover sufficiently fast the various manifestations of the disease, even though large amounts of data were accumulated in many hospitals EHRs worldwide. Herein, the present application and its embodiments are directed to a system for effective annotation of EHRs. This system will enable automatic annotation of large amounts of EHR text blocks currently residing in EHRs where they are inaccessible for research.

The embodiments of the present application comprise a complex process composed of several stages. This allows one to curate a specialized interface terminology for a given medical discipline, e.g., cardiology. For example, one can populate this Cardiology Interface Terminology (CIT) with concepts mined from cardiology EHRs. This ensures the effective annotation of cardiology EHRs with the CIT. The same process can be applied for any specific medical specialty. The nature of the described solution, however, does not preferably allow dealing with all medical specialties together, since the resulting interface terminology required would be too large.

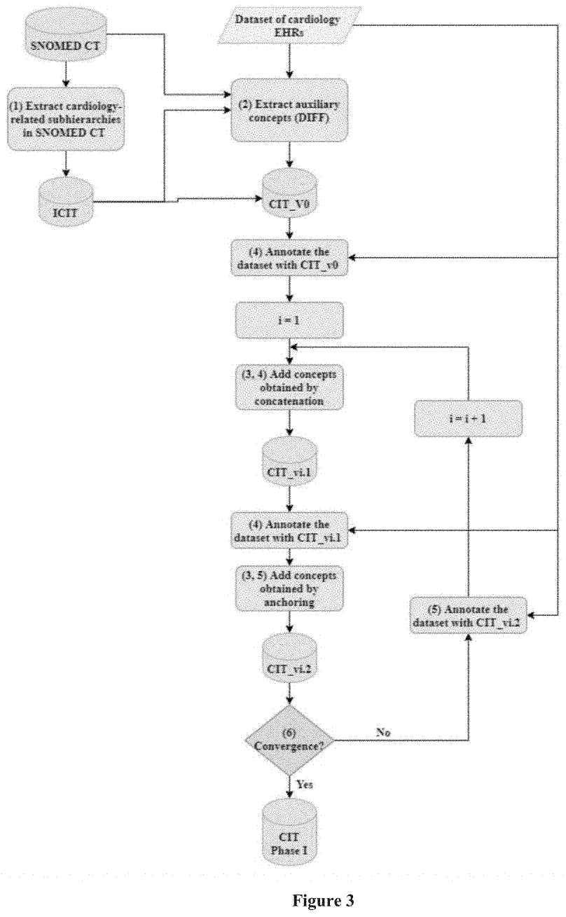

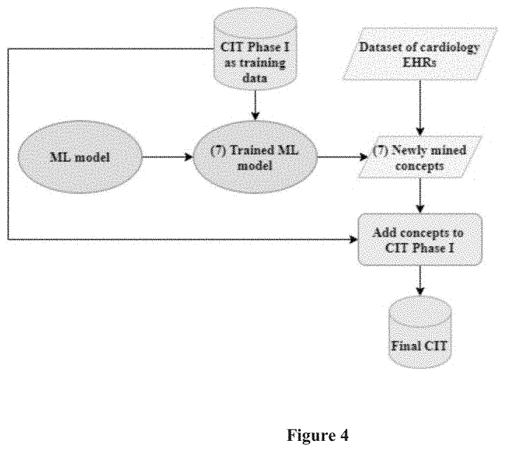

The preferred embodiment of the present application comprises at least the following phases, illustrated by the two flowcharts in FIGS. 3 and 4 describe Phase 1 and Phase 2 of the process, respectively.

In order to obtain an initial CIT one needs to identify the cardiology-related subhierarchies of SNOMED CT, containing concepts relevant to cardiology. The initial CIT consists of those subhierarchies. (For the COVID-19 disease, the initial CIT consists of concepts from several existing specialized COVID-19 ontologies, since SNOMED does not contain enough COVID-19 related concepts; other processes may be used for other indications.)

In Phase 1 of the annotation process, one must “enrich” the CIT via initial data collection. Preferably, one will annotate large samples of cardiology EHRs, first with SNOMED and then again with the initial CIT (ICIT). A DIFF (difference) operation between those two annotations identifies concepts that are not cardiology-related concepts but are typically appearing in the text of cardiology EHRs. Those describe generic terms for symptoms, diagnoses, treatments, and medications related to the medical history of a cardiology patient. Other kinds of concepts identified by the DIFF operation relate to non-medical terms, e.g., time-related concepts required when describing patient histories in EHRs. Those concepts identified by the DIFF operation are added to the initial CIT as auxiliary hierarchies to create CIT_v0 (version 0).

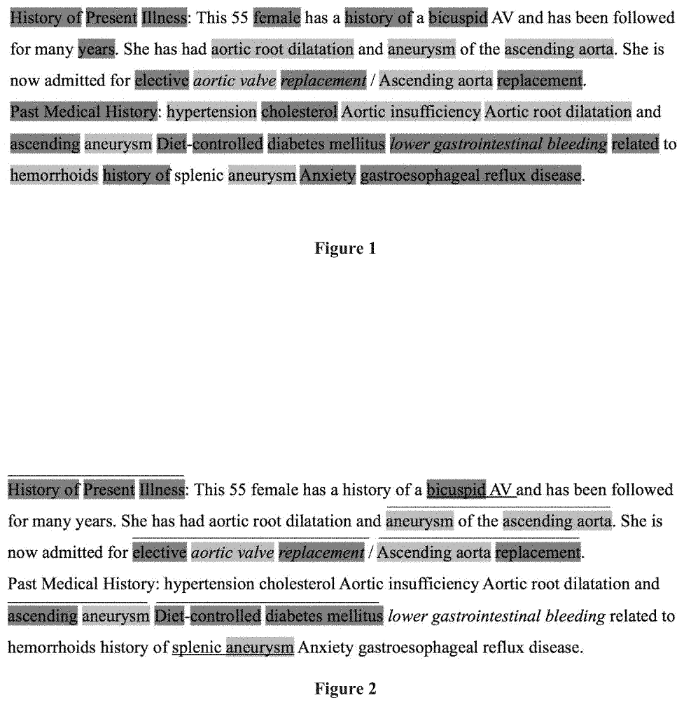

FIG. 1 illustrates the impact of such enrichment to create CIT_v0. The concepts of ICIT are highlighted in light grey, and the concepts obtained from SNOMED by the DIFF operation are highlighted in dark grey. In order to enhance the CIT_v0 with concepts that are refined cardiology-related concepts (in short: cardiology concepts) and are not in SNOMED, one may look for phrases in cardiology EHRs, since those will likely be best suited for annotating cardiology EHRs. Many such phrases contain, as a subunit, a concept that does appear in SNOMED. To automate the mining of such phrases from EHRs, the embodiments of the present application innovatively utilize two operations called “concatenation” and “anchoring”.

In concatenation, two concepts of CIT_v0, which are in the EHR text, and potentially connected by one or more stop words (in, the, to, an, below, . . . ) are combined into a new candidate concept. In anchoring, one is to attach to the phrase expressing a CIT_v0 concept occurrence in the EHR text either a word before, or a word after, or both, in the HER text connected potentially with one or more stop words to obtain a new candidate concept. FIG. 2., for example, demonstrates concepts obtained by concatenation (marked by overbars) or anchoring (marked by underlines) of annotated concepts in highlighted grey (either light or dark). The process of enhancement of the CIT is an iterative process, since a refined concept might be obtained by an alternating sequence of concatenation and anchoring operations.

After annotating some amount of EHRs with CIT_v0, the system, via a processor, is automatically able to generate all phrases findable in the EHR text by concatenation of CIT_v0 concepts. The phrases or candidate concepts obtained may then be reviewed by domain experts (or others) for their fitness as CIT concepts. Following this review, approved phrases are added as concepts to CIT_v1,1.

After annotating this EHR text with CIT_v1,1, the system, via a processor, is automatically able to generate all phrases obtainable by anchoring of CIT_v0 concepts. The phrases obtained are reviewed by domain experts (or others) for their fitness as CIT concepts. Following this review, approved phrases are added to create CIT_v1,2. This process is replicated as necessary for creating CIT_v2,1 , CIT_v2,2 , CIT_v3,1, CIT_v3,2, etc. until the number of new concepts added in the last iteration is smaller than a variable threshold (e.g. <50). Falling below such a threshold will always happen, but is dependent to a degree on the number of replications required. New concepts can only be generated from concepts added in the previous version since those generated in earlier versions were already added. Thus, as shown in FIG. 3, the process is said to “converge” which then directs the system to Phase 2 thereafter.

Phase 2 begins with the CIT obtained by this convergence serving as the initial cardiology interface terminology for curation based on machine learning (ML) techniques. As is understood in the art, ML techniques require training data. Phase 1 of the process described herein occurs first because no such training data was available for adding EHR phrases into an interface terminology. Applying ML techniques trained with the concepts added to CIT by concatenation and/or anchoring operations, the system can enhance the CIT with concepts, pending expert review, that are expressed by additional phrases mined from EHR notes, and which were not obtainable solely with anchoring and concatenation.

The final CIT obtained after applying the ML techniques is fit for use for effective automatic annotation of cardiology EHRs. The process of creating the CIT is preferably done only once. The resulting CIT can be used for the automatic annotation of an unlimited number of EHRs thereafter.

### Performance Metrics

Defined herein are two performance metrics: 1) “coverage” is the percentage of words being annotated, and 2) “breadth” is the average number of words per annotated concept.

Coverage=100*#annotated words/#all words

Breadth=#annotated words/#annotated concepts

The general rationale for the coverage metric is that the higher the coverage is, the higher the amount of meaningful clinical information captured from the EHR will be. For example, the coverage of the excerpt in FIG. 1 is 19/76=25% with ICIT (light grey), and 48/76=63% with CIT_V₀. (both light and dark grey)

The rationale for the breadth metric is that longer phrases help to better convey a chunk describing clinical information. For example, the chunk elective aortic valve replacement, obtained by concatenation in FIG. 2, with breadth 4. It conveys the intended clinical information much better than before the concatenation (in FIG. 1) with a breadth value of 4/3=1.33. Thus, capturing chunks rather than discrete individual concepts increases breadth.

In Table 1, we show the progression of the values of the coverage and the breadth metrics during the process of constructing the CIT for a dataset DS_{build} of 134 EHR case studies of COVID-19 patients from Radiopaedia[25] in a recent study[24]. Since there is no sufficient coverage of COVID-19 in SNOMED the initial CIT (ICIT) is obtained by integration of available COVID-19 ontologies. The largest of them is COVID-19 Infectious Disease Ontology (CIDO). For comparison Table 1 presents also results for CIDO and SNOMED.

TABLE 1
COVERAGE AND BREADTH FOR ITERATIONS
OF CIT FOR COVID-19 EHR DATASET

| Version | # Concepts | Coverage | Breadth |
|---|---|---|---|
| CIDO | 7834 | 10.49% | 1.13 |
| SNOMED_CT | 354178 | 46.94% | 1.18 |
| ICIT | 12036 | 21.36% | 1.14 |
| CIT_V₀ | 12697 | 53.73% | 1.17 |
| CIT_V_{1.1} | 13156 | 55.51% | 1.38 |
| CIT_V_{1.2} | 13970 | 61.75% | 1.78 |
| CIT_V_{2.1} | 14166 | 62.33% | 2.19 |
| CIT_V_{2.2} | 14430 | 66.34% | 2.30 |
| CIT_V_{3.1} | 14594 | 66.82% | 2.35 |
| CIT_V_{3.2} | 14658 | 67.43% | 2.37 |
| CIT_V_{4.1} | 14686 | 67.50% | 2.39 |
| CIT_V_{4.2} | 14686 | 67.59% | 2.40 |

For annotating with CIDO alone, there was an obtained coverage of only 10.49% since the text of EHR notes of COVID patients did not have a high percentage of COVID-related terms. The breadth was 1.13 because most annotated concepts consisted of one word. For the ICIT, which integrates six COVID ontologies and adds COVID-related concepts from several general terminologies, 4202 concepts were added to the concepts of CIDO. This expansion approximately doubled the coverage to 21.36%.

In contrast, with the large clinical terminology SNOMED, with 354,178 concepts, a coverage of 46.96% was obtained. The much higher coverage was obtained, because SNOMED contains medical concepts, e.g., medical conditions such as COVID-19 comorbidities and medications that are not necessarily COVID related but appear, for example, in the medical history of a patient. In addition, SNOMED also contains general English concepts used in EHR notes, e.g., time-related concepts. The breadth is only slightly higher (1.18), because most of the SNOMED concepts, which appear in the dataset consist of one word.

In CIT_V₀, the ICIT concepts were added to concepts of the DIFF between SNOMED and ICIT. Thus, all the 661 concepts of SNOMED that appear in DS_{build} the EHR dataset used to enhance the CIT, but do not appear in ICIT were inserted into CIT_V₀. As a result, the coverage of DS_{build} for the CIT_V₀, at 53.73%, is higher than both individual coverages of SNOMED and ICIT.

With alternating applications of concatenation and anchoring, meaningful increases in the first two iterations were achieved. The increases in the two following iterations were low. During the fourth iteration, only 28 (<50) concepts were added to CIT; thus, convergence was achieved, and the process stopped with CIT=CIT_v4,2. The final coverage for DS_{build} is 67.59% and the breadth is 2.4.

From the data in Table 1, it is shown that concatenation iterations lead to a small increase in the coverage but a large increase in the breadth while anchoring iterations display the opposite phenomenon. This difference stems from the nature of these two operations. In concatenation, the concatenated words were annotated before so the only potential gain for coverage comes from annotation of stop-wards between the concatenated words in the EHR text. However, the number of words per concepts increases. In anchoring iterations, at least one word is added to the coverage with each anchoring operation. Thus, the coverage increases more in such iterations. The breadth is also increasing, but not as much as with concatenations where there are more cases of concatenation of more than two concepts.

### Automatic Enhancement of the Annotation Coverage for DSₜₑₛₜ

Further tested was the generalizability on a hold-out dataset DSₜₑₛₜ, which consist of 34 random EHR case studies of the same collection from Radiopaedia, and compared the coverage to the coverage obtained for the dataset DS_{build} used to build the CIT.

Since the CIT is enriched by phrases mined from DS_{build}, it is natural that the annotation coverage of DS_{build} with CIT will be increased after several iterations. The question is what coverage will be achieved by annotating DSₜₑₛₜ by the same version of the CIT.

The problem is with the SNOMED concepts which appear in DSₜₑₛₜ but were not yet added to the CIT_v0 since they did not appear in DS_{build}, because those SNOMED concepts in DSₜₑₛₜ that appear also in DS_{build} were already identified and added to CIT_V₀ in the beginning of the process, and thus are annotated by CIT. Therefore, we can perform the DIFF operation for automatically identifying these concepts. To obtain this difference DIFF′, the DSₜₑₛₜ is annotated, first with SNOMED and then again with CIT. DIFF′ is then obtained as the set difference between the two annotations of DSₜₑₛₜ. The concepts of DIFF′ are added to CIT to obtain CIT′. In this way, there is an increase in the annotation coverage for DSₜₑₛₜ by using CIT.

As was seen above, the annotation with the final version of the CIT achieved a coverage of DS_{build} that was higher by 21% than the coverage of DS_{build} with SNOMED. The coverage obtained for DSₜₑₛₜ was 11.23% for CIDO, 21.52% for ICIT and 46.74% for SNOMED. For those three terminologies, the coverages for DS_{build} and DSₜₑₛₜ are similar, since those three terminologies do not depend on DS_{build}. Interestingly, the results are close for CIT_V₀, where the coverage is 53.33% for DSₜₑₛₜ vs 53.73% for DS_{build}, even though the DIFF was extracted from DS_{build} and may have missed SNOMED concepts that appeared only in DSₜₑₛₜ. Apparently, most of the concepts of SNOMED in DSₜₑₛₜ were also in DS_{build}.

The final annotation for DSₜₑₛₜ was performed by CIT obtaining a coverage of 59.46%, and a breadth of 1.68. In comparison, for DS_{build} , the final CIT obtained a coverage of 67.59% and a breadth of 2.4. Hence, the techniques employed achieved for DSₜₑₛₜ a coverage that is about 88% of the coverage obtained for DS_{build}. Even though CIT was built by extracting concepts from DS_{build} nevertheless, it was quite effective for annotation of DSₜₑₛₜ with CIT which was obtained by enriching CIT with DIFF′. The reason for the difference in the breadth values is that DIFF′, which contains concepts of SNOMED that were added to CIT, consists of short SNOMED concepts in contrast to the longer concepts of CIT created by concatenation and anchoring operations.

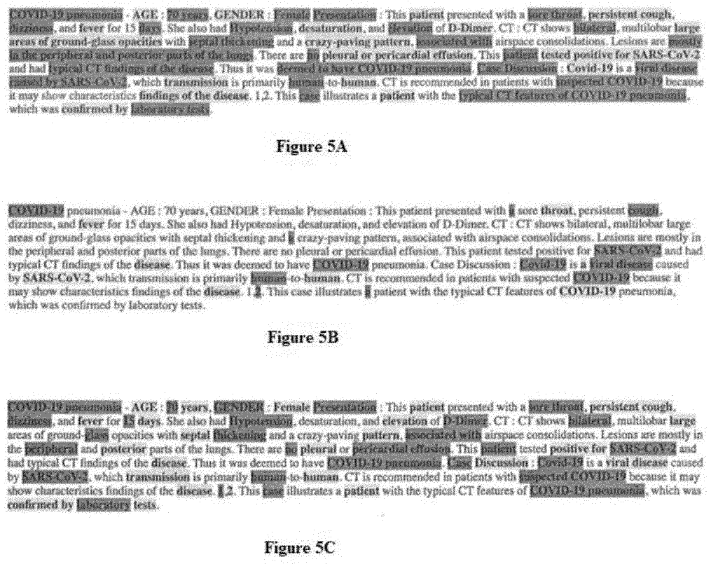

To illustrate the capacity of an annotated text to capture the content of an EHR note, we present a note from the test dataset DSₜₑₛₜ with annotation coverage level which is close to the average annotation coverage obtained for the test dataset with the CIT′. Furthermore, to emphasize the difference in the capability of various terminologies to capture the content of the clinical note we show in FIGS. 5A-C, the annotation of the same note with CIT′, CIDO and SNOMED in parts (A), (B) and (C) respectively. The reader can try to read only the annotated text and assess to what extent it is reflecting the content of the clinical note. FIGS. 5A-C use the light and dark grey colors alternatingly to mark the annotated phrases to easily distinguish between consecutive phrases.

In FIG. 5B showing the annotation with CIDO, only a few words, mostly with phrases related to COVID-19, are annotated. The annotated text fails to communicate the content of the note. The situation in FIG. 5C, however, is much better. A meaningful portion of the content is captured with the SNOMED concepts, but some parts of the text, especially parts related to the lung CT images are not captured. The annotation is still not capturing the complete content in a satisfactory fashion.

In contrast, in FIG. 5A, which shows annotation with CIT′ a substantial portion of the content of the clinical note is captured in the annotated phrases. In a numeric fashion it is manifested by the difference in the coverage metrics, 60.53% versus 41.45%. Consider some examples: “large areas of ground-glass opacities” is an annotated concept of CIT′ capturing the chunk by which a radiologist describes a CT image typical for COVID patients. In contrast, the SNOMED annotation captures only two isolated words “large” and “glass” of this chunk. Another example is “crazy paving pattern” which is a concept of CIT′, but only “pattern” from this chunk is annotated for SNOMED. At the same time, we acknowledge many phrases which are captured also by SNOMED e.g., “suspected COVID-19” and “COVID-19 pneumonia”. This example demonstrates why effective annotation with 75-80% is needed for performing research of textual notes. it also explains why, without available tools for effective annotation, EHRs in US research hospitals are mostly left unannotated.

### Expert Review of Candidate Phrases for CIT Concepts

In the description of the above process, it was mentioned that candidate phrases obtained by concatenation or anchoring are reviewed by domain experts who will decide whether they fit for inclusion in the CIT. These experts require both domain expertise and an understanding of terminologies. This manual review is time-consuming, however, it occurs only in the process of curating CIT, and once CIT is ready the annotation using an annotator is performed automatically.

To illustrate the review process, listed in Table 2 (below) from the study [24] of COVID-19 EHRs are examples of accepted concepts. In the top half of the table, listed are those concepts created by concatenation where each concatenated concept is enclosed with ‘|’ to the left and right of the concept name. The last concatenation example contains a stop word. The bottom half of the table contains examples of concepts obtained by anchoring. The anchor concepts are shown in bold font. The last example contains two stop words.

TABLE 2
EXAMPLES OF ACCEPTED PHRASES FROM CIT

| Version | Accepted Phrases |
|---|---|
| CIT_V_{1.1} | \|widespread\| \|bilateral\| \|consolidation\| |
| (Concatenation) | \|microvascular\| \|dilation\| |
| | \|extensive\| \|lung\| \|damage\| |
| | \|left\| \|upper\| \|paratracheal lymphadenopathy\| |
| | \|diffuse\| \|bilateral\| \|consolidation\| |
| | \|opacification\| in \|both lungs\| |
| CIT_V_{1.2} | atelectatic bands |
| (anchoring) | peripheral predominance |
| | crazy-paving pattern |
| | air bronchograms |
| | subpleural fibrous streak |
| | airspace opacification in the lungs |

In Table 3, shown below, also extracted from [24], presented are examples of phrases rejected during the review process. The major cause for the rejection of phrases was that the phrases generated are not complete. For example, |cases| of |mild| generated by concatenation (row 3) is a partial phrase (ending with an adjective and missing a noun to its right) corresponding to the complete phrase “cases of mild disease.” it is further demonstrated how similar examples of incomplete phrases generated by anchoring (in bottom half of Table 3). For anchoring, since it can be added to a word to the left, right or both in generating potential phrases, in some cases we obtain both partial and complete phrases in the same iteration. For example, the phrases Congested central, central vessels and Congested central vessels are generated by using central as the anchor concept, of which the first phrase is partial and hence rejected (Table 3).

TABLE 3
EXAMPLES OF REJECTED PHRASES

| Procedure | Rejected Phrases |
|---|---|
| Concatenation | \|patient\| had \|positive\| |
| | \|small\| \|foci of\| |
| | \|cases\| of \|mild\| |
| | \|bilateral\| \|mid\| |
| | \|endotracheal tube\| and \|right\| |
| Anchoring | disease in terms |
| | particularly at lower |
| | SARS-CoV-2 and the typical |
| | Congested central |
| | segments of both upper |
| | arched shape and occupies |

### An Alternative Approach

Above we describe how to overcome the problem that the result of the DIFF operation between SNOMED and ICIT is different if it is applied to DS_{build} or DSₜₑₛₜ. We present now an alternative solution for this problem. Instead of mining the necessary concepts of SNOMED from a dataset, to migrate, from SNOMED into CIT, complete subhierarchies of SNOMED which will contain all the potential concepts which may occur in a cardiology EHR dataset. This will include top levels of the Finding hierarchy of SNOMED which will contain non-cardiology medical conditions that may be reported in the medical history of a patient. Another subhierarchy of frequently prescribed medication. A third subhierarchy will contain all the time dependent concepts, etc.

The advantage of this approach is that we do not need to be concerned with the difference between datasets. The tradeoff is that this approach will generate a much larger CIT of a higher magnitude. Performing annotation with a terminology of a higher magnitude is considerably slower and annotation will take more time.

The present invention and its embodiments include both approaches to solve the problem. Depending on the setting, experiments will show which approach is preferred for each setting.

This research in [24] was done with a relatively small dataset. We expect higher coverage when a much larger dataset will be used, because for a larger dataset, CIT will capture more common phrases used in EHR notes of cardiology patients. Furthermore, we expect the gap between the coverages of DS_{build} and DSₜₑₛₜ to become smaller for a larger dataset because in such large datasets more common phrases are expected.

Hence utilizing large datasets to curate CIT, we expect a larger annotation coverage of about 75-80%.

Once CIT is curated, the annotation of unlimited number of cardiology EHRs is done automatically using e.g., the NCBO or any other annotator which can annotate with any terminology and thus also with CIT.

Such annotation will capture most of the relevant knowledge of the EHR notes and will enable extensive research into the rich knowledge hidden in the unstructured EHR notes of cardiology patients. However, the same is true for other disciplines, once this process is performed to obtain an interface terminology for that discipline.

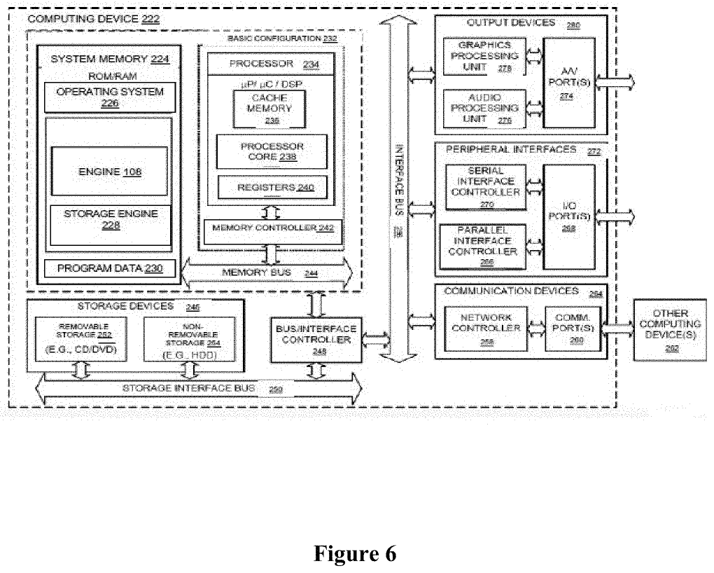

Referring now to FIG. 6, there is a block diagram of a computing device included within the system that is configured to implement one or more methods described herein, in accordance with embodiments of the present invention.

In some embodiments, the present invention may be a computer system, a method, and/or the computing device 106 or the computing device 222 (of FIG. 6). A basic configuration 232 of a computing device 222 is illustrated in FIG. 6 by those components within the inner dashed line. In the basic configuration 232 of the computing device 222, the computing device 222 includes a processor 234 and a system memory 224. In some examples, the computing device 222 may include one or more processors and the system memory 224. A memory bus 244 is used for communicating between the one or more processors 234 and the system memory 224.

Depending on the desired configuration, the processor 234 may be of any type, including, but not limited to, a microprocessor (μP), a microcontroller (μC), and a digital signal processor (DSP), or any combination thereof. Further, the processor 234 may include one more levels of caching, such as a level cache memory 236, a processor core 238, and registers 240, among other examples. The processor core 238 may include an arithmetic logic unit (ALU), a floating point unit (FPU), and/or a digital signal processing core (DSP Core), or any combination thereof. A memory controller 242 may be used with the processor 234, or, in some implementations, the memory controller 242 may be an internal part of the memory controller 242.

Depending on the desired configuration, the system memory 224 may be of any type, including, but not limited to, volatile memory (such as RAM), and/or non-volatile memory (such as ROM, flash memory, etc.), or any combination thereof. The system memory 224 includes an operating system 226, one or more engines, such as an engine 108, and program data 230. In some embodiments, the engine 108 may be an application, a software program, a service, or a software platform, as described infra. The system memory 224 may also include a storage engine 228 that may store any information disclosed herein.

Moreover, the computing device 222 may have additional features or functionality, and additional interfaces to facilitate communications between the basic configuration 232 and any desired devices and interfaces. For example, a bus/interface controller 248 is used to facilitate communications between the basic configuration 232 and data storage devices 246 via a storage interface bus 250. The data storage devices 246 may be one or more removable storage devices 252, one or more non-removable storage devices 254, or a combination thereof. Examples of the one or more removable storage devices 252 and the one or more non-removable storage devices 254 include magnetic disk devices (such as flexible disk drives and hard-disk drives (HDD)), optical disk drives (such as compact disk (CD) drives or digital versatile disk (DVD) drives), solid state drives (SSD), and tape drives, among others.

In some embodiments, an interface bus 256 facilitates communication from various interface devices (e.g., one or more output devices 280, one or more peripheral interfaces 272, and one or more communication devices 264) to the basic configuration 232 via the bus/interface controller 256. Some of the one or more output devices 280 include a graphics processing unit 278 and an audio processing unit 276, which are configured to communicate to various external devices, such as a display or speakers, via one or more A/V ports 274.

The one or more peripheral interfaces 272 may include a serial interface controller 270 or a parallel interface controller 266, which are configured to communicate with external devices, such as input devices (e.g., a keyboard, a mouse, a pen, a voice input device, or a touch input device, etc.) or other peripheral devices (e.g., a printer or a scanner, etc.) via one or more I/O ports 268.

Further, the one or more communication devices 264 may include a network controller 258, which is arranged to facilitate communication with one or more other computing devices 262 over a network communication link via one or more communication ports 260. The one or more other computing devices 262 include servers, the database, mobile devices, and comparable devices.

The network communication link is an example of a communication media. The communication media are typically embodied by the computer-readable instructions, data structures, program modules, or other data in a modulated data signal, such as a carrier wave or other transport mechanism, and include any information delivery media. A “modulated data signal” is a signal that has one or more of its characteristics set or changed in such a manner as to encode information in the signal. By way of example, and not limitation, the communication media may include wired media (such as a wired network or direct-wired connection) and wireless media (such as acoustic, radio frequency (RF), microwave, infrared (IR), and other wireless media). The term “computer-readable media,” as used herein, includes both storage media and communication media.

It should be appreciated that the system memory 224, the one or more removable storage devices 252, and the one or more non-removable storage devices 254 are examples of the computer-readable storage media. The computer-readable storage media is a tangible device that can retain and store instructions (e.g., program code) for use by an instruction execution device (e.g., the computing device 222). Any such, computer storage media is part of the computing device 222.

The computer readable storage media/medium can be a tangible device that can retain and store instructions for use by an instruction execution device. The computer readable storage media/medium may be, for example, but is not limited to, an electronic storage device, a magnetic storage device, an optical storage device, an electromagnetic storage device, and/or a semiconductor storage device, or any suitable combination of the foregoing. A non-exhaustive list of more specific examples of the computer readable storage media/medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and/or a mechanically encoded device (such as punch-cards or raised structures in a groove having instructions recorded thereon), and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Aspects of the present invention are described herein regarding illustrations and/or block diagrams of methods, computer systems, and computing devices according to embodiments of the invention. It will be understood that each block in the block diagrams, and combinations of the blocks, can be implemented by the computer-readable instructions (e.g., the program code).

The computer-readable instructions are provided to the processor 234 of a general purpose computer, special purpose computer, or other programmable data processing apparatus (e.g., the computing device 222) to produce a machine, such that the instructions, which execute via the processor 234 of the computer or other programmable data processing apparatus, create means for implementing the functions/acts specified in the block diagram blocks. These computer-readable instructions are also stored in a computer-readable storage medium that can direct a computer, a programmable data processing apparatus, and/or other devices to function in a particular manner, such that the computer-readable storage medium having instructions stored therein comprises an article of manufacture including instructions, which implement aspects of the functions/acts specified in the block diagram blocks.

The computer-readable instructions (e.g., the program code) are also loaded onto a computer (e.g. the computing device 222), another programmable data processing apparatus, or another device to cause a series of operational steps to be performed on the computer, the other programmable apparatus, or the other device to produce a computer implemented process, such that the instructions, which execute on the computer, the other programmable apparatus, or the other device, implement the functions/acts specified in the block diagram blocks.

Computer readable program instructions described herein can also be downloaded to respective computing/processing devices from a computer readable storage medium or to an external computer or external storage device via a network (e.g., the Internet, a local area network, a wide area network, and/or a wireless network). The network may comprise copper transmission cables, optical transmission fibers, wireless transmission, routers, firewalls, switches, gateway computers, and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer/computing device, partly on the user's computer/computing device, as a stand-alone software package, partly on the user's computer/computing device and partly on a remote computer/computing device or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to block diagrams of methods, computer systems, and computing devices according to embodiments of the invention. It will be understood that each block and combinations of blocks in the diagrams, can be implemented by the computer readable program instructions.

The block diagrams in the Figures illustrate the architecture, functionality, and operation of possible implementations of computer systems, methods, and computing devices according to various embodiments of the present invention. In this regard, each block in the block diagrams may represent a module, a segment, or a portion of executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block and combinations of blocks can be implemented by special purpose hardware-based systems that perform the specified functions or acts or carry out combinations of special purpose hardware and computer instructions.

Another embodiment of the invention provides a method that performs the process steps on a subscription, advertising, and/or fee basis. That is, a service provider can offer to assist in the method steps described herein. In this case, the service provider can create, maintain, and/or support, etc. a computer infrastructure that performs the process steps for one or more customers. In return, the service provider can receive payment from the customer(s) under a subscription and/or fee agreement, and/or the service provider can receive payment from the sale of advertising content to one or more third parties.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

### REFERENCES

[1] Blumenthal D. Stimulating the adoption of health information technology. W. V. Med. J. 2009;105:28-30.

[2] EHR Incentive Programs: 2015 through 2017 (Modified Stage 2) Overview. https://www.cdc.gov/ehrmeaningfuluse/docs/CMS_Stage_3_MU_Overvie w_2015_2017.pdf, (accessed 24 Aug. 2020).

[3] Donnelly K. SNOMED-CT: The advanced terminology and coding system for eHealth. Stud. Health Technol. Inform. 2006;121:279-90.

[4] Spackman K A, Campbell K E. Compositional concept representation using SNOMED: towards further convergence of clinical terminologies. AMIA Annu. Symp. Proc. 1998:740-4.

[5] Miller G A. The magical number seven, plus or minus two: Some limits on our capacity for processing information. Psychol. Rev. 1956;63:81.

[6] Miñarro-Giménez J A, Martínez-Costa C, Karlsson D, Schulz S, Gøeg KR. Qualitative analysis of manual annotations of clinical text with SNOMED CT. PLoS One. 2018;13:e0209547.

[7] Tchechmedjiev A, Abdaoui A, Emonet V, Melzi S, Jonnagaddala J, Jonquet C. Enhanced functionalities for annotating and indexing clinical text with the NCBO Annotator. Bioinformatics (Oxford, England). 2018;34:1962-5.

[8] Demner-Fushman D, Rogers W J, Aronson A R. MetaMap Lite: an evaluation of a new Java implementation of MetaMap. J. Am. Med. Inform. Assoc. 2017;24:841-4.

[9] Savova G K, Masanz J J, Ogren P V, Zheng J, Sohn S, Kipper-Schuler K C, et al. Mayo clinical Text Analysis and Knowledge Extraction System (cTAKES): architecture, component evaluation and applications. J. Am. Med. Inform. Assoc. 2010;17:507-13.

[10] Bodenreider 0. The Unified Medical Language System (UMLS): Integrating biomedical terminology. Nucleic Acids Res. 2004;32:D267-D70.

[11] Rosenbloom S T, Miller R A, Johnson K B, Elkin P L, Brown S H. Interface terminologies: facilitating direct entry of clinical data into electronic health record systems. J. Am. Med. Inform. Assoc. 2006;13:277-88.

[12] Rosenbloom S T, Brown S H, Froehling D, Bauer B A, Wahner-Roedler D L, Gregg W M, et al. Using SNOMED CT to Represent Two Interface Terminologies. J. Am. Med. Inform. Assoc. 2009;16:81-8.

[13] Kanter A S, Wang A Y, Masarie F E, Naeymi-Rad F, Safran C. Interface terminologies: bridging the gap between theory and reality for Africa. Stud. Health Technol. Inform. 2008:27-32.

[14] Johnson A E W, Pollard T J, Shen L, Lehman L-wH, Feng M, Ghassemi M, et al. MIMIC-III, a freely accessible critical care database. Scientific Data. 2016;3:160035.

[15] Jonquet C, Shah N H, Musen M A. The open biomedical annotator. Summit on Translat Bioinforma.;2009:56-60.

[16] Aronson A R, Lang F-M. An overview of MetaMap: historical perspective and recent advances. J. Am. Med. Inform. Assoc. 2010;17:229-36.

[17] Whetzel P, Shah N, Noy N, Dai B, Dorf M, Griffith N, et al. BioPortal: Ontologies and integrated data resources at the click of a mouse. Nature Precedings. 2009.

[18] Wang Y, Wang L, Rastegar-Mojarad M, Moon S, Shen F, Afzal N, et al. Clinical information extraction applications: A literature review. J. Biomed. Inform. 2018;77:34-49.

[19] Reátegui R, Ratté S. Comparison of MetaMap and cTAKES for entity extraction in clinical notes. BMC Med. Inform. Decis. Mak. 2018;18:74-.

[20] Soysal E, Wang J, Jiang M, Wu Y, Pakhomov S, Liu H, et al. CLAMP—a toolkit for efficiently building customized clinical natural language processing pipelines. J. Am. Med. Inform. Assoc. 2018;25:331-6.

[21] Keloth V K, Zhou S, Einstein A, Elhanan G, Chen Y, Geller J, Perl, Y. (2020). Generating Training Data for Concept-Mining for an ‘Interface Terminology’ Annotating Cardiology EHRs. 1728-1735. 10.1109/BIBM49941.2020.9313435

[22] Keloth V K, Zhou S, Einstein A, Lindemann L, Elhanan G, Geller J, Perl, Y, “Mining Concepts for a COVID Interface Terminology for Annotation of EHRs,” 2020 IEEE International Conference on Big Data (Big Data), 2020, pp. 3753-3760, doi: 10.1109/BigData50022.2020.9377981.

[23] SIRM. COVID-19 Database. https://www.sirm.org/category/senza-categoria/covid-19/, (accessed Jun. 5, 2020).

[24] Keloth V K, Zhou S, Einstein A, Lindemann L, Zhang L, Elhanan G, Geller J, Perl Y, Mining of EHR for Interface Terminology Concepts for Annotating EHRs of COVID patients, (submitted for journal publication.

[25] Radiopeadia, (2020). https://radiopaedia.org/ (accessed Jun. 15, 2020).

What is claimed is:

1. A electronic health record (EHR) annotation method for annotation of EHRs for a given medical discipline (D), the method implementing a design of a specialized discipline interface terminology (DIT), designed for annotation of a plurality of EHRs of the given medical discipline (D).

2. The method of claim 1 wherein the method comprises two phases,
wherein a first phase is based on mining concepts from the EHRs of the given discipline (D) and adding the mining concepts, pending expert review, to the DIT, and
wherein the second phase uses a machine learning technique with the mining concepts added to the DIT of the first phase, as training data, for the machine learning technique to produce a final version of the DIT.

3. The method of claim 2 wherein the first phase comprises the step of:
extracting, from a reference terminology, subhierarchies of concepts pertaining to the given discipline (D), resulting in an initial DIT.

4. The method of claim 3 wherein a difference operation is applied to the resulting sets of the two annotations from a plurality of EHRs,
wherein a first annotation of the two annotations is performed with a SNOMED terminology, and wherein a second annotation of the two annotations is performed with the initial DIT terminology.

5. The method of claim 4 wherein the difference of the two annotations is added, via a processor, to an initial DIT to generate a current version of the DIT.

6. The method of claim 5 wherein concatenating and anchoring operations are performed, via a processor, alternatingly on the plurality of EHRs annotated with a current version of the DIT.

7. The method of claim 6 wherein the concatenation and anchoring operations are alternatingly repeated at least one time, adding each of the at least one of the concepts mined from the plurality of EHRs by the concatenation and anchoring operations, pending an expert review to the current version of the DIT.

8. The method of claim 7 wherein the concatenating and anchoring operations are alternatingly repeated until the number of new terms added to a generated DIT falls below a threshold.

9. The method of claim 8 wherein the second phase applies, via a processor, a machine learning technique to add concepts to the generated DIT.

10. The method of claim 9 wherein the machine learning technique is trained using the training data which comprises the concepts which were added to DIT in the concatenating and anchoring operations, the trained machine learning model is applied to mine from a plurality of EHRs additional concepts to be added to a final DIT, pending expert review.

11. The method of claim 10 wherein the final DIT will be used to automatically annotate unlimited amounts of EHRs of disciple (D), using any annotator software which is configured to annotate with any given terminology.
